(19) United States
(12) Patent Application Publication
(10) Pub. No.: US 2005/0261779 A1
(43) Pub. Date: Nov. 24, 2005

(54) Expansible rod-type prosthesis and external magnetic apparatus

(76) Inventor: Rudolf X. Meyer, Pacific Palisades, CA (US)

Correspondence Address: Rudolf X. Meyer, 16966 Livorno Drive, Pacific Palisades, CA, 90272, US

(21) Appl. No.: 10/713,668

(22) Filed: Nov. 17, 2003

(51) Int. Cl. A61F 2/28; A61B 17/72

(52) U.S. Cl. 623/23.47; 623/23.45; 623/18.12; 600/409; 606/63

(57) ABSTRACT

This disclosure is for a rod-type prosthesis that, once surgically implanted, can be expanded non-invasively and repeatedly by an externally applied magnetic field. It applies to patients with a bone disease of a limb where a section of the bone must be surgically removed. If the patient is still at a growing age, current medical practice requires repeated surgical interventions to periodically implant longer and longer rods to match the normal growth of the other limbs. Similarly, in the case of adult patients a repeated lengthening of the prosthesis is often required. The present invention avoids the need for these repeated surgical interventions since the rod prosthesis can be non-invasively lengthened when the need arises.

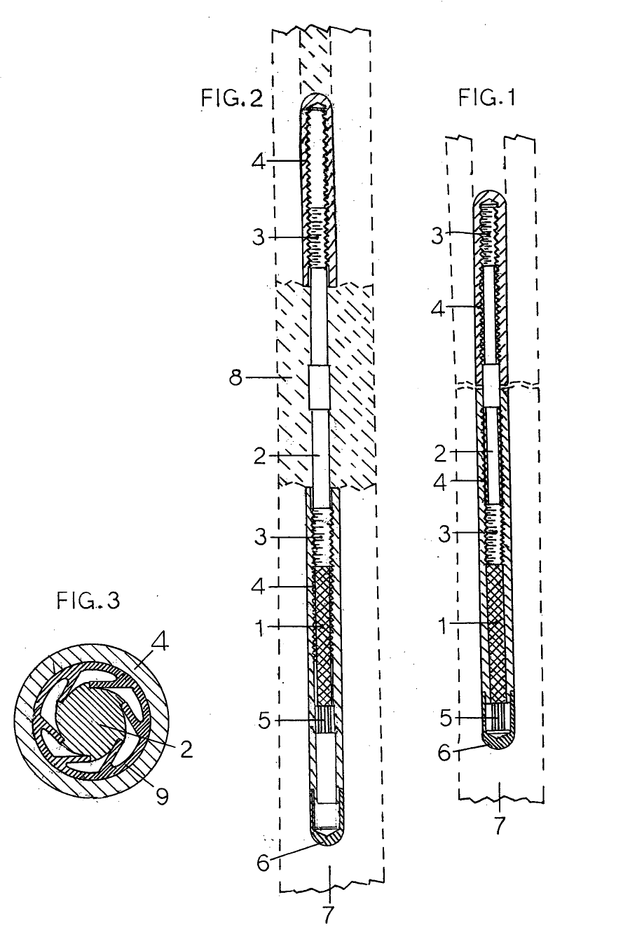

In FIG. 1 there is a single, rod-shaped permanent magnet designated by 1. Its direction of magnetization is perpendicular to its axis. Preferred type magnets are rare-earth permanent magnets, coated to be biocompatible. Other possible materials are platinum-cobalt alloys, or, less expensively, ferrites or Alnico. The shaft 2, which can be a Chrome-Nickel steel, is attached by adhesive to the permanent magnet 1 and has at its ends two screws 3 that engage the internal threads on the two tubular structures 4. The preferred material for these are titanium alloys or other non-ferromagnetic alloys that allow the external magnetic field from the external magnetic apparatus to penetrate to the permanent magnet of the prosthesis. The thread between 3 and 4 can be coated with Teflon or lubricated with a biocompatible lubricant such as graphite powder. 5 is a ratchet mechanism, shown enlarged in FIG. 3. The cap 6 serves to facilitate the assembly. The borie, for instance a femur, is shown in outline as 7.

FIG. 2 shows the same prosthesis as FIG. 1, but now in its fully expanded position. 1 to 7 are the same components as in FIG. 1. Where the new growth of the bone has occurred when the prosthesis has been expanded, is shown by 8. It is understood that the length of the prosthesis and therefore the length of the new growth can be anything between the extremes shown in FIGS. 1 and 2, respectively.

FIG. 3 is an enlarged section, taken perpendicular to the axis of the rod, of the ratchet mechanism that serves to prevent the rod from getting shorter when subjected to variable loads from motions by the patient. 2 is a continuation of the shaft and is connected by adhesive or similar means to the permanent magnet. 4 is part of the tubular structure that is also shown in FIGS. 1 and 2. The component 9 is an elastomer with prongs that engage 2. As shown, the combination of 2 and 9 permit rotation of 2 in a counter-clockwise direction that produces a lengthening of the prosthesis, but prevents rotation in the clockwise direction that would cause a shortening of the prosthesis. This type of ratchet is particularly well suited for prostheses with a small diameter, typical of rod-type prostheses. It is understood, however, that a conventional, metallic racket with spring loaded prongs could also be used.

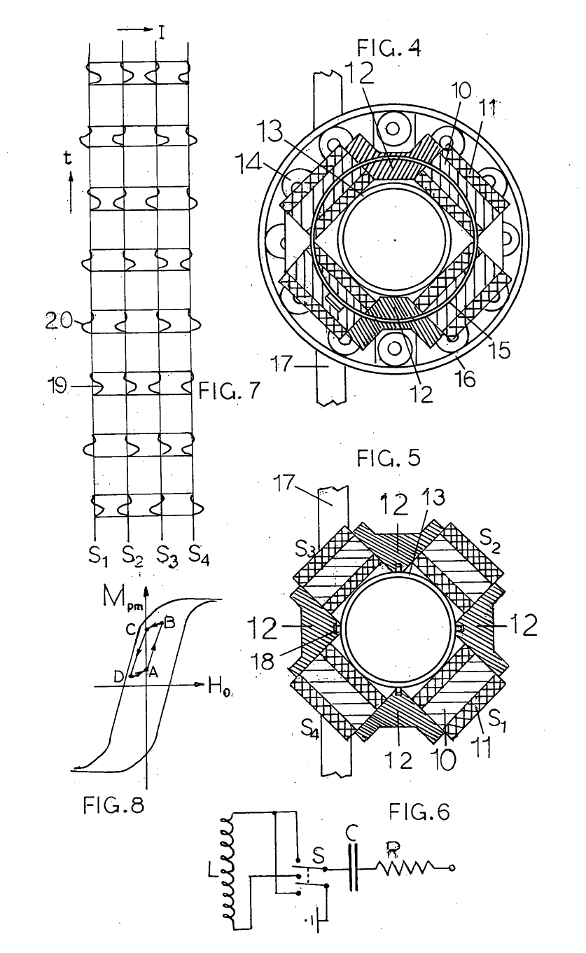

FIG. 4 is a section and view of the external magnetic apparatus, version 1, taken crosswise to the limb of the patient. This apparatus incorporates one or several electromagnets, powered with direct current, with soft iron cores 10 and electric windings 11. The soft iron pole pieces 12 produce the external magnetic field that interacts with the permanent magnet in the prosthesis. The directions of the current in the windings 11 are such as to produce a magnetic North in one of the pole pieces and South in the other. The tube 13 contains the limb of the patient. The assembly, other than 13, is supported by rollers 14 and can be rotated either by hand or by an electric motor around the limb. Slip rings 15 combined with conventional electric brushes that are stationary transmit the current from a stationary direct current power supply to the electromagnets. The rollers 14 run on a stationary ring 16 that is supported by the structural member 17 that is attached to a table on which rests the patient.

FIG. 5 represents a second version of the external magnetic apparatus. In this version, the electromagnets, having cores 10 and windings 11, are powered by short, but intense current pulses. There are at least four electromagnets, here labeled by S₁ to S₄. The directions of the currents in the winding change from pulse to pulse in such a manner that the same pole piece 12 can be a magnetic North or South. The effect of switching the current directions in the electromagnets is such as to produce an external magnetic field that rotates in space by 90 degrees, inducing a corresponding rotation of the permanent magnet in the prosthesis. The components 13 and 17 are the same as in FIG. 4. There are at four Hall-current sensors 18 that sense the magnetic field of the permanent magnet in the prosthesis and allow the physician to determine the rotational position of the permanent magnet in the prosthesis without need for x-ray examination. To avoid interference from the more intensive external magnetic field, these sensors are activated only between the current pulses.

Although somewhat more complicated than version 1, there are two advantages to version 2. First, the peak currents in a compact device can be much larger than the direct currents in version 1, resulting in a larger torque exerted on the permanent magnet in the prosthesis, which in turn produces a larger force available for the expansion. Because of the short duration of the pulses, with longer pauses between pulses, high peak currents can be tolerated without overheating the windings of the electromagnets. Second, the external magnetic apparatus is simplified since it does not require the mechanical rotation about the patient's limb.

FIG. 6 is an electrical circuit diagram that shows the charging, via a resistor R, and discharging of a capacitor C. The double switch S is either open, or closed to direct the current into the electromagnet L in one direction, or closed to direct the current in the opposite direction. Such circuits can be used separately for each of the electromagnets shown in FIG. 5.

FIG. 7 is a timing diagram for the current pulses, I, that activate the electromagnets S₁ to S₄ that are shown in FIG. 5. The letter t and the associated arrow indicate the time and its direction. The horizontal lines delineate the start and cut-off of the pulses by the switch S shown in FIG. 6. By example, 19 is defined as a positive pulse, 20 as a negative one. The sequence of pulses that is shown rotates the permanent magnet in the prosthesis in four steps of 90 degrees through a complete revolution. Each step of 90 degrees requires two sets of pulses (as will be clear from FIG. 8). Depending on the desired amount of expansion of the prosthesis, the sequence of pulses can be either shorter than shown, or can be repeated wholly or in part.

FIG. 8 is a schematic hysteresis diagram for a permanent magnet in the prosthesis. The abscissa H₀ is the magnetic field strength produced by the external magnetic apparatus at the center of the permanent magnet. The ordinate Mₚₘ is the (variable) magnetization of the permanent magnet. A set, at a given time, of the four current pulses to the four electromagnets will move a point in the diagram from A (at the beginning of the set of pulses) to B to C (at the end of the set of pulses). The next set of pulses will move the point from C to D and back to A. These pairs of sets of pulses can be repeated as is required by the desired amount of expansion of the prosthesis.

While particular forms of the invention have been illustrated and described, it will be apparent that various modifications can be made to the present invention without departing from the spirit and scope thereof

1. A prosthesis, in the form of a rod with a length much greater than its diameter, that is surgically implanted and can be expanded in length without new surgery, in combination with a magnetic apparatus that is external to the patient's body and generates a magnetic field that interacts with the magnet in the said prosthesis comprising:
a magnet in the said prosthesis;
a mechanism that transforms the motion of the said magnet in the prosthesis, relative to other parts of the said prosthesis, into an expansion of the length of the said prosthesis;
means to rotate the magnetic field that is generated by the said magnetic apparatus relative to the patient's body.

2. The combination defined in claim 1 wherein the said mechanism comprises at least one pair of mutually meshing male and female threads and wherein the said motion of the said magnet in the prosthesis is a rotation relative to other parts of the said prosthesis and causes an expansion of the said prosthesis.

3. The combination defined in claim 1 wherein the said magnet in the said prosthesis is a permanent magnet.

4. The combination defined in claim 1 wherein the said magnet in the said prosthesis is an electromagnet.

5. The combination defined in claim 1 wherein there is in the said prosthesis a ratchet mechanism that allows an expansion of the length of the said prosthesis, but prevents a reduction of the length.

6. The combination defined in claim 1 wherein the said magnetic apparatus is supported by rollers or by bearings that allow the rotation of the said magnetic apparatus around the patient's limb that contains the prosthesis.

7. The combination defined in claim 1 wherein the magnetic field of the said magnetic apparatus is produced by at least one electromagnet.

8. The combination defined in claim 1 wherein the magnetic field of the said magnetic apparatus is produced by at least one permanent magnet.

9. The combination defined in claim 7 wherein the electric power to the said electromagnets is supplied via slip rings and electric brushes from a stationary power supply.

10. The combination defined in claim 5 wherein the said ratchet mechanism contains an elastomer with flexible prongs that engage the teeth on the shaft of the said ratchet mechanism.

11. A magnetic apparatus external to a patient's limb and comprising at least four electromagnets, in combination with prostheses that use an internal magnet, the said magnetic apparatus generating magnetic fields interacting with the magnet in the prosthesis by means of current pulses that are short compared to the pauses between them.

12. The combination defined in claim 11 wherein the said current pulses are generated by discharges of capacitors.

13. The combination defined in claim 11 wherein electric switches control the length and timing of the said current pulses and, when needed, reverse the current direction in the said current pulses.

14. The combination defined in claim 1 wherein the said magnetic apparatus comprises at least one magnetic field sensor and readout for the observation by the physician of the position of the said magnet in the said prosthesis, obviating the need for multiple x-ray examinations.

15. The combination defined in claim 1 wherein a sling is provided that is exerting a controlled stretching force to the patient's limb that has the said prosthesis.
